(19) United States
(12) Patent Application Publication
(10) Pub. No.: US 2021/0322680 A1
(43) Pub. Date: Oct. 21, 2021

(54) Method and apparatus for implementing magnetic micro-syringes

(71) Applicant: Weinberg Medical Physics Inc, North Bethesda, MD (US)

(72) Inventors: Irving WEINBERG, North Bethesda, MD (US); Sahar JAFARI, North Bethesda, MD (US)

(21) Appl. No.: 17/232,285

(22) Filed: Apr. 16, 2021

Related U.S. Application Data
(60) Provisional application No. 63/011,720, filed on Apr. 17, 2020.

(51) Int. Cl. A61M 5/315

(52) U.S. Cl. CPC A61M 5/31531; A61M 2205/19; A61M 2205/0272

(57) ABSTRACT

Disclosed embodiments enable a method and apparatus of constructing and using magnetic micro-syringes for human or veterinary therapeutic or non-therapeutic procedures.

### CROSS REFERENCE AND PRIORITY CLAIM

This application relies for priority on U.S. Provisional Patent Application Ser. No. 63/011,720, entitled “METHOD AND APPARATUS OF MAGNETIC MICRO-SYRINGES” filed Apr. 17, 2020, the entirety of which is incorporated by reference.

### FIELD

Disclosed embodiments are directed, generally, to a method and apparatus of constructing and using magnetic micro-syringes for human or veterinary therapeutic or non-therapeutic procedures.

### BACKGROUND

Drugs can have unwanted effects if the drugs are delivered to the wrong tissues in a subject's/patient's body. Magnetic drug delivery is a promising drug delivery approach that uses magnetic fields to aim one or more drug-carrying vehicles at one or more target tissues or organs. Such magnetic drug-carrying vehicles must be able to carry and release payloads, where such payloads are understood to be drugs or other therapeutic molecules or moieties (for example, cells).

### SUMMARY

Disclosed embodiments provide a multi-segment payload-delivery vehicle in which one segment's purpose is primarily to load, carry, and release the payload and a method of constructing the same.

In accordance with at least one embodiment, an additional segment of the payload-carrying vehicle is devoted to propulsion and/or rotation of the vehicle under the influence of an applied magnetic field.

In accordance with at least one embodiment, the vehicle, described in this disclosure as a micro-syringe, may also be used to sample tissues.

### BRIEF DESCRIPTION OF FIGURES

FIG. 1 illustrates an embodiment of a vehicle containing at least one partially hollow segment intended primarily for loading, carrying, and releasing a payload, and at least one other attached segment containing magnetizable material intended primarily for propelling and/or rotating the apparatus;

FIG. 2 illustrates an embodiment of the vehicle in which segment used for propulsion and/or rotation is subdivided into smaller sub-segments;

FIG. 3 depicts a method of building the vehicle according to the disclosed embodiments;

FIG. 4 depicts a method of filling the vehicle constructed using the methods of FIG. 3; and

FIG. 5 illustrates a method of imaging and/or manipulating vehicle within the body.

### DETAILED DESCRIPTION

For the purposes of this disclosure, the vehicle may be termed a vehicle, particle, or micro-syringe. The term “micro-syringe” is not intended to imply a limit as to the size of parts or the entire particle, which may be in the nanometer range, the micrometer range, or the centimeter range. The term “vehicle” is intended to describe an example of the invented particle or the result of the invention method described herein. It is understood that in use, the invented apparatus may contain one or more such vehicles.

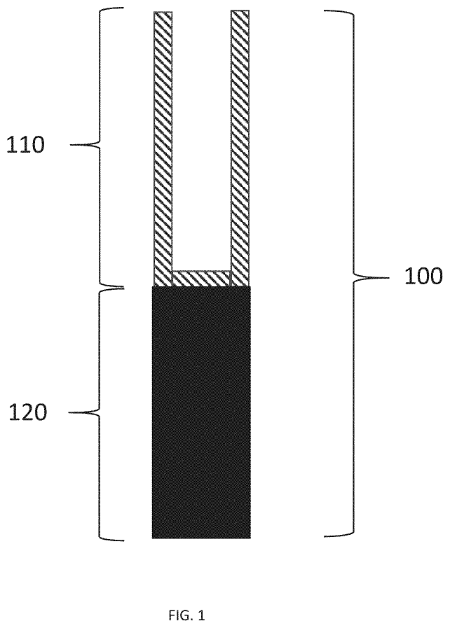

FIG. 1 illustrates an embodiment of a vehicle 100 containing at least one partially hollow segment 110 intended primarily for loading, carrying, and releasing a payload, and at least one other attached segment 120 containing magnetizable material intended primarily for propelling and/or rotating the apparatus. Segments 110 and 120 may be further comprised of sub-segments. The embodiments and methods described herein may comprise one or more such vehicles.

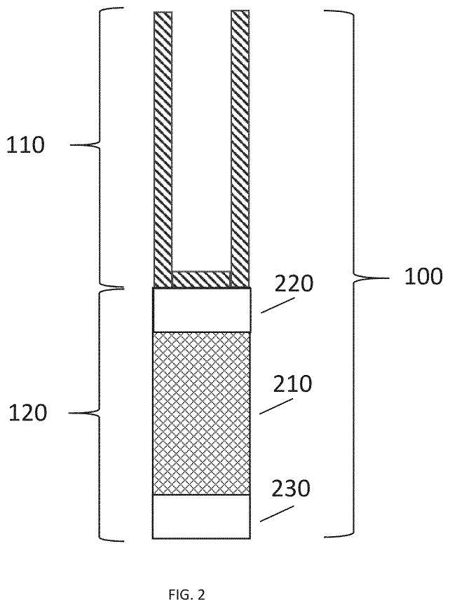

FIG. 2 illustrates an embodiment of the vehicle 100 in which segment 120 used primarily for propulsion and/or rotation is subdivided into smaller sub-segments, including magnetizable sub-segment 210 and non-magnetizable sub-segments 220 and 230.

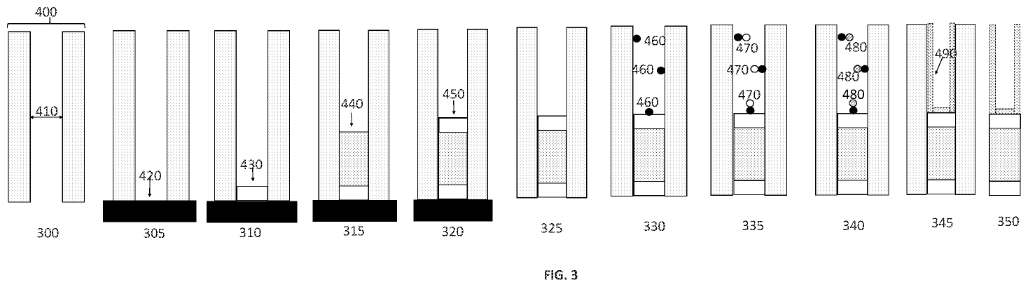

FIG. 3 illustrates a method of building the vehicle through successive operations 300-350, as discussed in detail below. In the first operation 300, a template 400 contains many through-holes (also described herein as “pores”). An example of one through-hole is shown as 410. It is understood that the operations of the method described below are applied simultaneously to many through-holes in template 400. Template 400 may be made of porous Anodic Aluminum Oxide (AAO), which is available commercially as a filtration membrane and features uniform pore diameters and between-pore distances, or may be made of other porous materials. In some embodiments, multiple templates 400 are processed through the methods described herein, each template on a conveyor belt or other holder. In some embodiments, multiple templates 400 comprise a belt that is conveyed from chamber to chamber for a roll-to-roll process. In some embodiments, the pore diameter is 250 nm. In other embodiments, pore 410 diameter may be as small as 10 nanometers or as large as 10 microns, or values in between these extremes. The apparatus is built up in the operations below is described as a structure, with the understanding that the structure corresponds to multiple structures being built in many pores 410 in template 400.

In the next operation 305, an electrically conductive backing layer 420 is deposited on one side of template 400 (for example by sputtering silver or copper onto the template). In some embodiments, backing layer 420 is 100 nm thick, but it may be as small as 5 nm and as large as 10 microns, or other values in between these extremes.

In the next operation 310, a non-magnetic (for example, gold) sub-segment 430 is electrodeposited onto the surface of the electrically conductive layer 420. In some embodiments, layer 430 is 100 nm thick, but it may be as small as 5 nm and as large as 10 microns, or other values in between these extremes.

In the next operation 315, a ferromagnetic (for example, iron) sub-segment 440 is electrodeposited onto the surface of the previously deposited layer 430. In some embodiments, layer 440 is 300 nm thick, but it may be as small as 5 nm and as large as 10 microns, or other values in between these extremes.

In the next operation 320, another non-magnetic (for example, gold) sub-segment 450 is electrodeposited onto the surface of the magnetic layer 440. In some embodiments, layer 450 is 100 nm thick, but it may be as small as 5 nm and as large as 10 microns, or other values in between these extremes.

Operations 315 and 320 may be repeated one or more times, possibly varying the thickness of deposited layers, in order to obtain multiple magnetic layers separated by non-magnetic layers. Operation 315 may be repeated, possibly varying the thickness of deposited layers, in order to obtain multiple adjacent magnetic layers, possibly using different magnetic materials. Multiple magnetic layers may be helpful when used in conjunction with appropriate external magnetic fields in order to spin and pull the apparatus, as taught in U.S. Pat. No. 10,290,404, entitled “Method and Apparatus for Non-Contact Axial Particle Rotation and Decoupled Particle Propulsion” incorporated by reference.

Thereafter, at 325, electrically conductive backing layer 420 is removed, for example by inserting the entire structure in 70% nitric acid for five minutes. In several next operations 330 through 350, hollow segments are formed in portions of the structure.

In operation 330, multiple silicon atoms 460 may be added to the exposed surface of pore 410 by immersing the structure described above for forty minutes at room temperature in a solution of 5% (by weight) of 3-triethoxysilyl propyl-succinic anhydride (95% in ethanol). Acetic acid (60.05 g/mol) may then be added to the above solution, in order to increase the acidity. The structure may then be annealed by placing it in an oven at 90° C. for two hours.

Subsequently, at 335, a coating of tin atoms 470 may be added to the silicon-coated surface of the pore 410. This addition may be done by immersing for forty--five minutes the structure in a solution containing SnCl2 (0.026 M) and trifluoroacetic acid (0.07 M). The solvent of the solution may be 50/50 methanol/water or maybe pure water. The structure may then be rinsed with water and then dried in air.

Thereafter, at 340, tin coating 470 may be oxidized from the Sn(II) state to the Sn(IV) state and replaced by silver 480, by immersing the structure for 30 minutes in silver nitrate (at a concentration of 0.029 M) with 1 ml NH4OH 35.05 g/mol). Silver 480 may remain as a coating of the structure while the tin goes into solution. The structure may then be rinsed with water and air-dried.

Subsequently, at 45, silver atoms 480 may be galvanically replaced by gold 490 by immersing the structure in a gold-plating bath for about 24 hours. The bath may be prepared by dissolving the following substances in 480 ml of water at approximately 3° C.: 6 ml of gold electroless plating solution, 1 gram of sodium sulfite, 12 ml of 37% formaldehyde and 0.6 gram of NaHCO3. The pH of the bath may be adjusted to 9 by adding 1M sulfuric acid drop-by-drop. The structure may then be rinsed with distilled water.

In the next operation 350, vehicles may be released from the template by immersing the structure in 2M solution of NaOH, which now include both magnetic segments and gold nanotube drug carrier segments. Particles may be rinsed with ethanol and may be sterilized and may then be placed in a sterile solution (for example, water or saline) in a vial or other container (not shown). In accordance with at least one embodiment, micro-syringe particles may be dried and placed in a vial or other container for later reconstitution or dilution.

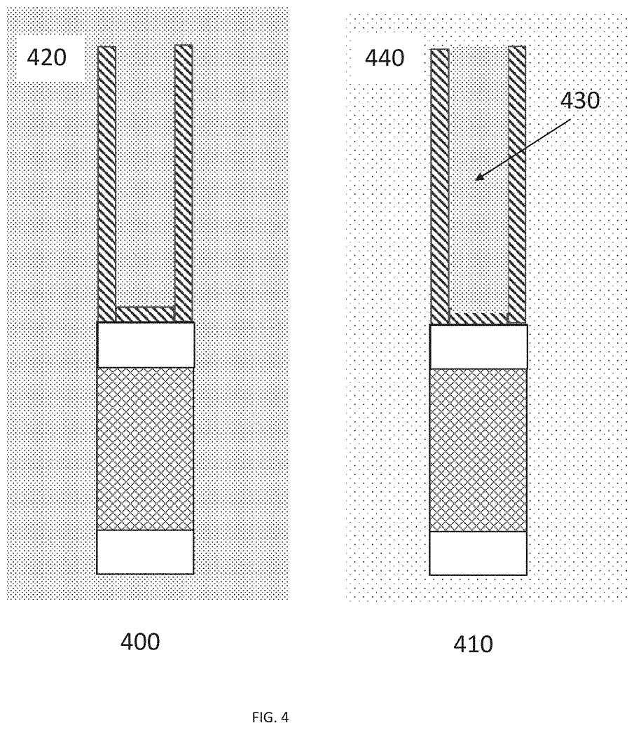

FIG. 4 shows a method of filling the vehicle constructed using the methods of FIG. 3. In operation 400, one more micro-syringe particles are immersed in a fluid 420 containing drugs, cells, genes, or other moieties of interest. This immersion may be accomplished by injecting the moiety of interest into a vial or other container containing one or more micro-syringe particles. The solution containing the moiety of interest may enter the hollow section of the micro-syringe through capillary action. The fluid 420 may be subsequently processed to be solid or porous. The micro-syringe particles may then be collected from the vial with the aid of a magnet or other operation (for example, centrifugation) and dried. In an embodiment, the loaded micro-syringe particle (loaded with moiety-containing fluid 430 may be suspended in another solution 440 (as in operation 410) and contained in a vial or administered to a human or animal for therapeutic or diagnostic purposes.

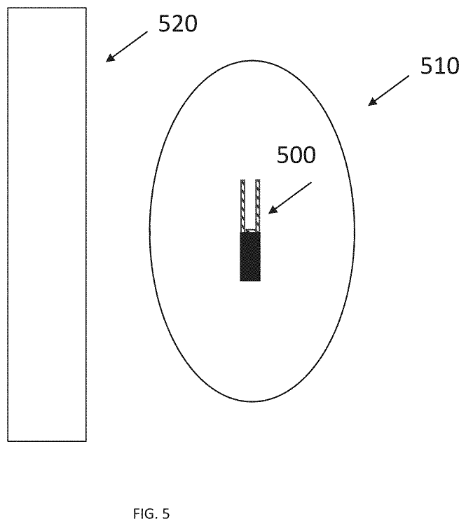

FIG. 5 illustrates method operations for imaging and/or manipulating vehicle 500 (which is the same as vehicle 100 in FIG. 1) within a body 510, utilizing an instrument 520 that may be sensitive to the presence of magnetic material, such as a Magnetic Resonance Imaging (MRI) instrument or Magnetic Particle Imaging (MPI) instrument. Instrument 520 may also have the ability to move or otherwise actuate vehicle 500, for example by applying magnetic fields to vehicle 500 before, during, or after vehicle 500 enters body 510.

It is understood that sub-segments 210, 220, 230 of magnetic segments 120 may be varied in number of sub-segments and size of each sub-segment, for example to achieve a desired effect in combination with magnetic fields applied to the micro-syringe particles whether in or outside of a body. The term “body” is meant to include the anatomic extent of a cell, human, or animal, whether healthy or not. The term “body” may also include an inanimate object, for example a body of water. The term “magnetic” as applied to a material is understood to mean ferromagnetic, paramagnetic, super-paramagnetic or magnetizable in any way.

For the purposes of this disclosure, the term “hollow” as applied to a vehicle or to a segment of a vehicle means that a substantial portion (for example, 50% or 90%) of the vehicle or segment of vehicle can be empty of liquids or solids (for example, the hollow portion containing only air) by a user or processor. This the vehicle described herein is different from other types of vehicles that carry payloads through incorporation of the payload into a solid material that is attached or part of the vehicle. A processor or user is defined as an individual or group of individuals wishing to use the hollow vehicle to carry a payload into, within, or from a body. The hollow section of the vehicle or segment may be partially or totally filled with fluid or cells or powders or other materials by the user or processor.

The vehicles 500 may be moved into, within, and from a body 510 using magnetic fields applied to the body by an instrument 520. It is therefore understood that the vehicles may be employed to sample tissues in a body. This may be useful, for example, in sampling regions in a body that are suspicious for the presence of tumorous cells or other undesirable conditions. It is understood that the vehicles may be employed to deliver chemicals, cells, drugs, genetic materials or other useful substances into, within, and from a body. It is understood that external magnetic and/or electromagnetic fields may actuate the vehicles. The term “actuation” implies the creation or modulation of effects on the vehicles, for example heating. The actuation may be synergistic with the delivery of drugs, for example particle heating (for example from the application of alternating magnetic fields) or rotation. Such synergy may include enhanced release of the payload under the influence of the effects. It is understood that additional or different operations during or after processing may be taken to cover the micro-syringe particles with other materials, for example a plastic coating that may delay delivery of the particle payload. The coating might be affected by the above synergistic actions to modify the rate of delivery, for example by not releasing the payload until the vehicle was at an appropriate location.

It is understood that the magnetic nature of portions of the vehicle may allow the particle's progress in the body to be tracked with an instrument sensitive to the presence of magnetic materials, such as an MRI instrument or an MPI instrument. It is understood that the vehicle may contain substances that are visible with other instruments, for example if the payload or portions of the vehicle are radioactive or have optical or sonographic properties such as fluorescence.

It should be understood that the operations explained herein may be implemented in conjunction with, or under the control of, one or more general purpose computers running software algorithms to provide the presently disclosed functionality and turning those computers into specific purpose computers.

Moreover, those skilled in the art will recognize, upon consideration of the above teachings, that the above exemplary embodiments may be based upon use of one or more programmed processors programmed with a suitable computer program. However, the disclosed embodiments could be implemented using hardware component equivalents such as special purpose hardware and/or dedicated processors. Similarly, general purpose computers, microprocessor based computers, micro-controllers, optical computers, analog computers, dedicated processors, application specific circuits and/or dedicated hard wired logic may be used to construct alternative equivalent embodiments.

Moreover, it should be understood that control and cooperation of the above-described components may be provided using software instructions that may be stored in a tangible, non-transitory storage device such as a non-transitory computer readable storage device storing instructions which, when executed on one or more programmed processors, carry out he above-described method operations and resulting functionality. In this case, the term “non-transitory” is intended to preclude transmitted signals and propagating waves, but not storage devices that are erasable or dependent upon power sources to retain information.

Those skilled in the art will appreciate, upon consideration of the above teachings, that the program operations and processes and associated data used to implement certain of the embodiments described above can be implemented using disc storage as well as other forms of storage devices including, but not limited to non-transitory storage media (where non-transitory is intended only to preclude propagating signals and not signals which are transitory in that they are erased by removal of power or explicit acts of erasure) such as for example Read Only Memory (ROM) devices, Random Access Memory (RAM) devices, network memory devices, optical storage elements, magnetic storage elements, magneto-optical storage elements, flash memory, core memory and/or other equivalent volatile and non-volatile storage technologies without departing from certain embodiments. Such alternative storage devices should be considered equivalents.

1. An apparatus comprising:
at least one vehicle including a plurality of segments,
wherein at least one magnetic segment of the plurality of segments is magnetic and at least one segment of the plurality of segments is hollow.

2. The apparatus of claim 1, further comprising an instrument external to a body, wherein the instrument is sensitive to the magnetic material in the at least one vehicle.

3. The apparatus of claim 2, wherein the instrument is configured to move or otherwise actuate the at least one vehicle into, within, or out of the body using magnetic or electromagnetic propulsion.

4. The apparatus of claim 1, wherein the at least one hollow segment is configured to carry and release a payload.

5. The apparatus of claim 1, wherein the at least one magnetic segment comprises a plurality of magnetic segments, each separated by a non-magnetic segment.

6. The apparatus of claim 5, wherein the at least one hollow segment is formed at an end of the instrument distal to the plurality of magnetic segments.

7. The apparatus of claim 6, wherein the at least one hollow segment is a nanotube.

8. A method of constructing at least one vehicle, the method comprising:
successive deposition within a porous template of one or more magnetic materials and of one or more coatings prior to release of the at least one vehicle from the porous template, wherein upon release of the vehicle from the template the vehicle contains at least one magnetic segment and at least one hollow segment.

9. The method of claim 8, wherein the one or more magnetic layers and the one or more coatings are applied via electrodeposition.

10. The method of claim 8, wherein the one or more coatings comprise non-magnetic materials.

11. The method of claim 10, wherein the thickness of the one or more magnetic layers and one or more non-magnetic materials is varied.

12. The method of claim 8, wherein the at least one hollow segment is formed subsequent to the successive deposition of the one or more magnetic materials and one or more coatings on a distal end of the at least one vehicle.

13. The method of claim 8, wherein the at least one hollow segment is formed in the template as a nanotube.

14. The method of claim 13, wherein releasing the at least one vehicle from the template comprises submerging the template with the at least one vehicle in a solution of NaOH.

15. The method of claim 13, further comprising filling the at least one hollow section with a payload by submerging the released at least one vehicle in a solution including the payload.

16. A method of using an instrument external to a body, the method comprising:
moving at least one vehicle into, within, or out of a body,
wherein the at least one vehicle includes a plurality of segments,
wherein at least one segment of the plurality of segments is magnetic and at least one segment of the plurality of segments is hollow.

17. The method of claim 16, wherein an instrument external to a body is sensitive to the at least one magnetic segment in the at least one vehicle.

18. The method of claim 17, wherein the at least one magnetic segment comprises a plurality of magnetic segments, each separated by a non-magnetic segment.

19. The method of claim 18, wherein the instrument is configured to move or otherwise actuate the vehicle into, within, or out of the body using magnetic or electromagnetic propulsion.

20. The method of claim 16, where in the at least one hollow segment is configured to carry a payload.
